(19) United States
(12) Patent Application Publication
(10) Pub. No.: US 2005/0020904 A1
(43) Pub. Date: Jan. 27, 2005

(54) System and method for the detection of brain iron using magnetic resonance imaging

(76) Inventors: Harvey Ellis Cline, Niskayuna, NY (US); Ronald Dean Watkins, Niskayuna, NY (US); John Frederick Schenck, Voorheesville, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 10/617,543

(22) Filed: Jul. 10, 2003

(51) Int. Cl. A61B 5/055

(52) U.S. Cl. 600/410; 600/420

(57) ABSTRACT

A method and system for detecting iron using magnetic resonance imaging (MRI) is provided. The method comprises generating a substantially high magnetic field strength within the MRI system, acquiring magnetic resonance (MR) images by a pulse sequence adapted to create a magnetic field map of the brain for use in enhancing brain iron deposits, and, characterizing regions of interest using the magnetic field map to detect statistically relevant quantities of brain iron deposits to indicate a given disease.

### BACKGROUND OF THE INVENTION

The invention relates to magnetic resonance imaging (MRI) and image processing methods. More particularly, the invention relates to detection of brain iron using MRI and image processing techniques.

It has been known for some time that specific regions of the brain contain deposits of iron in a storage pool consisting of iron atoms in a mineral matrix associated with and largely surrounded by associated proteins. The total complex of mineralized iron and proteins is referred to as ferritin or in other cases as hemosiderin. It has also been recognized that these deposits are to some extent capable of being visualized on MR images because of the tendency of the magnetized iron atoms to alter the local magnetic field and to thereby to reduce the MR signal from protons in water molecules and other compounds in their vicinity of the iron deposits. This effect is referred to as iron-dependent shortening of the local T2 relaxation time. It is known that this effect is more prominent and more easily observed at higher magnetic field strengths. However, this imaging phenomenon has not been widely used for diagnostic purposes because of the difficulty in making diagnostic inferences due to the limited sensitivity of standard MR scanners and the complex and irregular shapes of the affected brain regions. Consequently, there is a need for an invention to improve the sensitivity of MR imaging to the presence of brain iron deposits and to improve the methods of analysis of the MR images to detect disease-related changes. One urgent need in neurology is an imaging method capable of detecting abnormal deposits in the brain, such as amyloid plaques and neurofibrillary tangles, which are associated with Alzheimer's disease and related diseases. It is known that iron in the form of ferritin or related proteinaceous compounds is often associated with these deposits. Although these deposits are often too small to be imaged as individual structures within the brain by MRI, the presence of several such deposits within an MR imaging voxel may lead to reduced overall signal strength for this voxel because of the iron content. Thus, by a process of signal averaging across a single voxel, this technique may be used to establish the presence of these pathological structures. Furthermore, a number of degenerative brain diseases (e.g., Parkinson's disease, Hallervorden Spatz disease and many others) have been found to be associated with increased regional iron deposition.

To date, most efforts to utilize brain-iron dependent contrast have utilized relatively thick slice (e.g., 3-5 mm), low-field (e.g., 1.5 T) images analyzed by visual inspection or by measurements of the image intensity variation or T2 relaxation of individual voxels. This method is cumbersome and time-consuming and, unless high-resolution imaging is used, local details of the iron distribution are not resolved.

The susceptibility of the brain is influenced by small quantities of magnetic material such as iron. The magnetic resonance susceptibility contrast mechanism involves phase changes induced by local variations in magnetic field. Since the resonance frequency is proportional to the magnetic field and the phase of an image depends on the echo time times the frequency. A phase image may be used to map the magnetic field.

The magnetic field may vary due to the instrument inhomogenuity and the magnetism of the patient being imaged. The long ranges variations of the instrument may be removed by shimming in a well-known manner wherein a set of shim coils are excited to compensate for the magnetic field variation. The currents applied to the shim coils are estimated by sampling the phase image of the patient in the region of interest. The slowly varying terms in 3D may be represented by a spherical harmonic series.

Thus, there is a need for methods to perform MR imaging of brain iron deposition for use in the diagnosis of and monitoring the progression of neuro-degenerative brain diseases that overcome the deficiencies and problems described above. More particularly, there a need for improved sensitivity of MR imaging to detect the presence of brain iron deposits and to improve the methods of analyzing MR images to diagnose disease and detect disease related changes.

### BRIEF SUMMARY OF THE INVENTION

In a first aspect, a method for detecting iron in the brain using magnetic resonance imaging (MRI) is provided. The method for detecting iron in the brain using magnetic resonance imaging (MRI) comprises generating a substantially high magnetic field strength within the MRI system, acquiring magnetic resonance (MR) images by a pulse sequence adapted to create a magnetic field map of the brain for use in enhancing brain iron deposits, and, characterizing regions of interest within using the magnetic field map to detect statistically relevant quantities of brain iron deposits to indicate a given disease.

In a second aspect, a system for detecting iron in the brain using magnetic resonance imaging (MRI) is provided. The system a magnetic resonance imaging device having a substantially high magnetic field strength and the device being adapted for acquiring magnetic resonance (MR) images by a pulse sequence adapted to create a magnetic field map of the brain for use in enhancing brain iron deposits, and, an image processor coupled to the imaging device and adapted for characterizing regions of interest using the magnetic field map to detect iron deposits for use in at least one of diagnosis, prognosis, and prediction of progression of iron-dependent diseases.

### BRIEF DESCRIPTION OF THE DRAWINGS

The features and advantages of the present invention will become apparent from the following detailed description of the invention when read with the accompanying drawings in which:

FIG. 1 illustrates a simplified block diagram of a Magnetic Resonance Imaging system to which embodiments of the present invention are useful; and,

FIG. 2 is an exemplary illustration of MR images of brain iron taken at a magnetic field strength of 3 Tesla (3 T) to which embodiments of present invention are applicable.

### DETAILED DESCRIPTION OF THE INVENTION

MRI scanners, which are used in various fields such as medical diagnostics, typically use a computer to create images based on the operation of a magnet, a gradient coil assembly, and a radio frequency coil(s). The magnet creates a uniform main magnetic field that makes nuclei, such as hydrogen atomic nuclei, responsive to radio frequency excitation. The gradient coil assembly imposes a series of pulsed, spatial-gradient magnetic fields upon the main magnetic field to give each point in the imaging volume a spatial identity corresponding to its unique set of magnetic fields during the imaging pulse sequence. The radio frequency coil(s) creates an excitation frequency pulse that temporarily creates an oscillating transverse magnetization that is detected by the radio frequency coil and used by the computer to create the image.

Generally, very high field strength is characterized as greater than 1.5 Tesla (1.5 T). In recent years, there has been an increase in usage of MRI systems at field strengths above the typical 1.5 Tesla. Research systems have been built as high as 8 Tesla. Systems are now commercially available at 3 Tesla and 4 Tesla. The systems are primarily used for research in functional MRI (fMRI) and human head related imaging and spectroscopy studies.

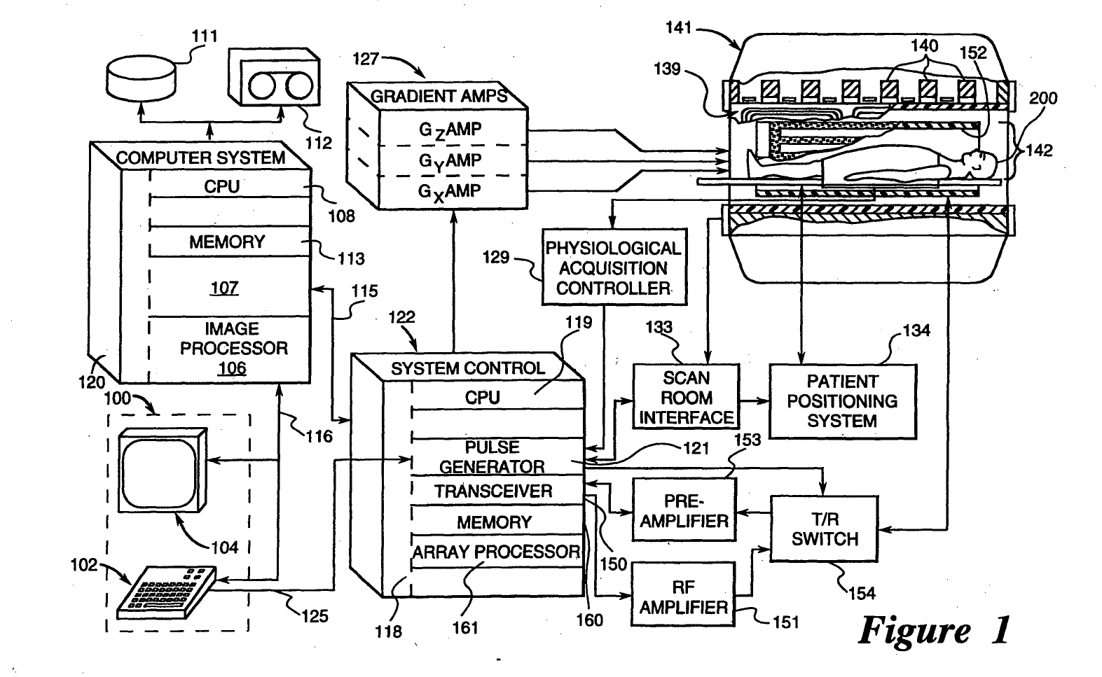

FIG. 1 illustrates a simplified block diagram of a system for producing images in accordance with embodiments of the present invention. In an embodiment, the system is a MR imaging system which incorporates the present invention. The MRI system could be, for example, a GE-Signa MR scanner available from GE Medical Systems, Inc., which is adapted to perform the method of the present invention, although other systems could be used as well.

The operation of the MR system is controlled from an operator console 100 which includes a keyboard and control panel 102 and a display 104. The console 100 communicates through a link 116 with a separate computer system 107 that enables an operator to control the production and display of images on the screen 104. The computer system 107 includes a number of modules that communicate with each other through a backplane. These include an image processor module 106, a CPU module 108, and a memory module 113, known in the art as a frame buffer for storing image data arrays. The computer system 107 is linked to a disk storage 111 and a tape drive 112 for storage of image data and programs, and it communicates with a separate system control 122 through a high speed serial link 115.

The system control 122 includes a set of modules connected together by a backplane. These include a CPU module 119 and a pulse generator module 121 which connects to the operator console 100 through a serial link 125. It is through this link 125 that the system control 122 receives commands from the operator which indicate the scan sequence that is to be performed. The pulse generator module 121 operates the system components to carry out the desired scan sequence. It produces data that indicate the timing, strength, and shape of the radio frequency (RF) pulses that are to be produced, and the timing of and length of the data acquisition window. The pulse generator module 121 connects to a set of gradient amplifiers 127, to indicate the timing and shape of the gradient pulses to be produced during the scan. The pulse generator module 121 also receives subject data from a physiological acquisition controller 129 that receives signals from a number of different sensors connected to the subject 200, such as ECG signals from electrodes or respiratory signals from a bellows. And finally, the pulse generator module 121 connects to a scan room interface circuit 133 (which receives signals from various sensors associated with the condition of the subject 200) and the magnet system. It is also through the scan room interface circuit 133 that a positioning device 134 receives commands to move the subject 200 to the desired position for the scan.

The gradient waveforms produced by the pulse generator module 121 are applied to a gradient amplifier system 127 comprised of Gₓ, G_{y} and G_{z} amplifiers. Each gradient amplifier excites a corresponding gradient coil in an assembly generally designated 139 to produce the magnetic field gradients used for position encoding acquired signals. The gradient coil assembly 139 forms part of a magnet assembly 141 which includes a polarizing magnet 140 and a whole-body RF coil 152. Volume 142 is shown as the area within magnet assembly 141 for receiving subject 200 and includes a patient bore. As used herein, the usable volume of a MRI scanner is defined generally as the volume within volume 142 that is a contiguous area inside the patient bore where homogeneity of main, gradient and RF fields are within known, acceptable ranges for imaging. A transceiver module 150 in the system control 122 produces pulses that are amplified by an RF amplifier 151 and coupled to the RF coil 152 by a transmit/receive switch 154. The resulting signals radiated by the excited nuclei in the subject 200 may be sensed by the same RF coil 152 and coupled through the transmit/receive switch 154 to a preamplifier 153. The amplified MR signals are demodulated, filtered, and digitized in the receiver section of the transceiver 150. The transmit/receive switch 154 is controlled by a signal from the pulse generator module 121 to electrically connect the RF amplifier 151 to the coil 152 during the transmit mode and to connect the preamplifier 153 during the receive mode. The transmit/receive switch 154 also enables a separate RF coil (for example, a head coil or surface coil) to be used in either transmit or receive mode. As used herein, “adapted to”, “configured” and the like refer to mechanical or structural connections between elements to allow the elements to cooperate to provide a described effect; these terms also refer to operation capabilities of electrical elements such as analog or digital computers or application specific devices (such as an application specific integrated circuit (ASIC)) that is programmed to perform a sequel to provide an output in response to given input signals.

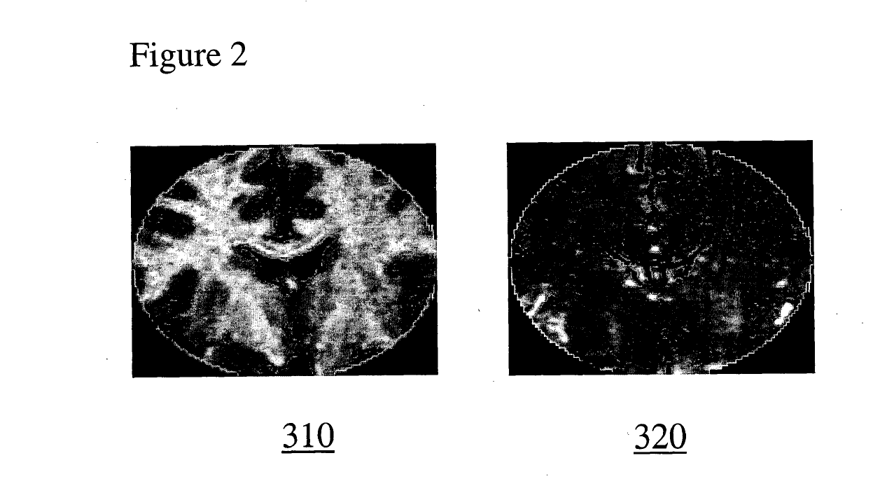

The MR signals picked up by the RF coil 152 are digitized by the transceiver module 150 and transferred to a memory module 160 in the system control 122. When the scan is completed and an entire array of data has been acquired in the memory module 160, an array processor 161 operates to Fourier transform the data into an array of image data. These image data are conveyed through the serial link 115 to the computer system 107 where they are stored in the disk memory 111. In response to commands received from the operator console 100, these image data may be archived on the tape drive 112, or they may be further processed by the image processor 106 and conveyed to the operator console 100 and presented on the display 104. Image processor 106 is further adapted to perform the image processing techniques which will be in greater detail below and with reference to FIG. 2. It is to be appreciated that a MRI scanner is designed to accomplish field homogeneity with given scanner requirements of openness, speed and cost.

As used herein, the term “very high field” refers to magnetic fields produced by the MRI system that are greater than about 1.5 Tesla. For embodiments of the invention the high field is desirably about 3 Tesla (3 T). Also, as used herein, “very high frequency” is considered to be the range of about 64 MHz to about 500 MHz, with a desired range between about 128 MHz and about 300 MHz. For embodiments of the invention, the high frequency is desirably at about 128 MHz.

All data gathered from multiple scans of the patient is to be considered one data set. Each data set can be broken up into smaller units, either pixels or voxels. When the data set is two-dimensional, the image is made up of units called pixels. A pixel is a point in two-dimensional space that can be referenced using two-dimensional coordinates, usually x and y. Each pixel in an image is surrounded by eight other pixels, the nine pixels forming a three-by-three square. These eight other pixels, which surround the center pixel, are considered the eight-connected neighbors of the center pixel. When the data set is three-dimensional, the image is displayed in units called voxels. A voxel is a point in three-dimensional space that can be referenced using three-dimensional coordinates, usually x, y and z. Each voxel is surrounded by twenty-six other voxels. These twenty-six voxels can be considered the twenty-six connected neighbors of the original voxel.

In an exemplary embodiment of the present invention, high-resolution MR images are taken preferably at a magnetic field strength of 3 Tesla or more using a three-dimensional (3D) gradient dual echo pulse sequence to acquire image data at two different echo times such that a 3D phase image is acquired. These images may use a slice thickness of 1.5 mm or less. Alternatively, it to be appreciated by one skilled in the art that any pulse sequence may be used that is configured to acquire a 3D phase image or alternatively to create a magnetic field map of the region of interest (brain). As used herein, the term “magnetic field map” refers to measurements acquired during MRI to estimate the constant and linear components of the magnetic field inhomogeneity. Pulse generator module 121 is adapted to produce the 3D phase images and magnetic field maps as described herein.

In embodiments of the present invention, flattening of the magnetic field in the region of interest is simulated by fitting a spherical harmonic series to a three dimensional phase image. For example, a 3D gradient echo sequence may be acquired at two different echo times and than the phase difference represents the magnetic field map. To fit a spherical harmonic series of N terms to the magnetic field map of the patient's brain a well known numerical method is used. Samples of the field B(Xm,Ym,Zm) in the region of interest are taken over a volume at the coordinates (Xm,Ym,Zm) where m=1 . . . M. The values of the N spherical harmonic functions Fn(Xm,Ym,Zm) at the sample points form an N by M matrix. The coefficients Cn of the spherical harmonic series are given by a matrix equation [F][C]=[B] which is solved by linear numerical methods to give a best least square fit to the data. The smoothed spherical harmonic series Sum [Cn times Fn(X,Y,Z)] is subtracted from the measured magnetic field map giving the local variation in magnetic field in the volume of interest. The equation is provided as:
Local Field Variation=B(X,Y,Z)−Sum(Cn Fn(X,Y,Z)

The variation in the magnetic field increases with the magnitude to the magnetic field. B0 and the signal to noise in MRI also increases linearly with B0. Thus the ability to measure magnetic field variations in the brain with MRI increases as B0 squared. Thus, high field MRI using embodiments of the present invention advantageously provide a more sensitive measure of brain magnetism and iron that is involved with neurological disease.

In an embodiment of the present invention, a method for detecting iron in the brain using magnetic resonance imaging (MRI) comprises the steps of acquiring magnetic resonance (MR) images by using high magnetic field strength and a pulse sequence adapted to create a magnetic field map of the brain and then fitting of the magnetic field map to a spherical harmonic series to create a smoothed map for enhancing brain iron deposits for use in characterizing the regions of interest using the magnetic field maps to detect statistically relevant quantities of brain iron deposits to indicate a given disease. Generally, brain iron deposits are associated and indicative Alzheimer's disease, Parkinson's disease, Huntington's disease, Hallervorden Spatz disease, other neurodegenerative disorders, and other diseases of the central nervous system. Depending on the disease, there may be more or less statistically relevant brain iron to characterize the given disease. In an alternative embodiment, the characterizing of brain iron comprises measuring MR signal modifications produced by the brain deposits and using the signal modification in monitoring at least one of the progression of a given disease and response to therapeutic activity. Further, characterizing the brain iron comprises processing the regions of interest using computer-aided analysis based on image intensity, T2 values, intensity ratios and signal loss in order to enhance detection of brain iron within brain substructures. Additionally, characterizing further comprises producing volumetric measurements of the regions of interest, wherein the volumetric measurements are used in quantifying progression of the given disease and/or monitoring response to therapy.

In a further embodiment, the steps of acquiring and characterizing are repeated in at least one successive or serial examination, typically at a later time, of a given subject for measuring progression of the disease and measuring response to therapy. Additionally, the method includes interfacing with a data source, such as same subject examination data, clinical population data for the given disease and bioinformatic data, in order for the image processor to perform comparisons of the regions of interest with data from the respective data sources. As more and more is known about neurodegenerative disease and corresponding relevant iron information, then comparison with the data sources would enable disease staging, predictive modeling and other such tracking of the disease for a given patient.

A number of degenerative brain diseases (e.g., Parkinson's disease, Hallervorden Spatz disease and many others) have been found to be associated with increased regional iron deposition. With the high resolution M imaging and computer analysis, as described herein, it is likely that many new brain regions with high iron depositions will be identified and characterized, thereby extending this diagnostic technique to additional disease states. Furthermore, the use of computer-generated information, such as volumetric analysis of affected brain regions and the ability to track this parameter in serial studies of a given patient by use of computer image registration techniques, provides a means of quantifying the progression of disease and the response to therapy.

Once image data is acquired and analyzed by the process described above, the image data may be used for various aspects of disease diagnosis and tracking. For example, quantitative characterization of iron deposits will enable a physician to track the disease progression or response to therapy of a patient. The acquisition and characterization are repeated and patient image data can be followed serially in a given patient through the use of image registration techniques. Another advantage is the possibility of quantifying the spatial extent and intensity of iron-deposition in and thereby providing quantitative volumetric measures of irregularly shaped brain nuclei. The method provides a convenient, computer-assisted tracking of changes in iron deposition associated with disease onset, progression and therapy.

FIG. 2 shows an exemplary illustration of MR images of brain iron taken at a magnetic field strength of 3 Tesla (3 T) to which embodiments of present invention are applicable. The magnitude image 310 on the left shows a region of the brain in a coronal slice at the level of the lateral ventricle. The phase image 320 shows the magnetic field variation using spherical harmonics to remove the slow variations. The contrast is related to tissue magnetism. For example, the concentration of iron would influence the tissue susceptibility. Thus, through the use of methods in accordance with the present invention described above, it is possible to detect brain iron within brain structures which provides the ability to diagnose and detect disease related changes.

Embodiments described above focused on methods to enhance the detection of brain iron for the purpose of diagnosing and detecting neurodegenerative diseases. However, it is to be appreciated that the methods of the present invention would be similarly applicable to imaging structures outside the brain, for example the liver. One skilled in the art would find the methods of acquiring and characterizing to enhance iron deposits could be applied similarly to diseases such as hereditary hemochromatosis and secondary hemochromatosis which lead to an iron overload in the liver and other tissues.

While the preferred embodiments of the present invention have been shown and described herein, it will be obvious that such embodiments are provided by way of example only. Numerous variations, changes and substitutions will occur to those of skill in the art without departing from the invention herein. Accordingly, it is intended that the invention be limited only by the spirit and scope of the appended claims.

1. A method for detecting iron in the brain using magnetic resonance imaging (MRI) comprising:
generating a substantially high magnetic field strength within the MRI system;
acquiring magnetic resonance (MR) images by a pulse sequence adapted to create a magnetic field map of the brain for use in enhancing brain iron deposits; and,
characterizing regions of interest using the magnetic field maps to detect statistically relevant quantities of brain iron deposits to indicate a given disease.

2. The method of claim 1 wherein the selected pulse sequence is a dual gradient echo pulse sequence and the acquiring step is performed for two different echo times.

3. The method of claim 1 wherein the acquiring step generates a three dimensional phase image of the brain.

4. The method of claim 3 wherein the magnetic field map of the brain is created by fitting a spherical harmonic series to the three dimensional phase image of the brain.

5. The method of claim 4 further comprises subtracting the spherical harmonic series from the magnetic field map to provide a measure of local variation in the magnetic field in the brain.

6. The method of claim 1 wherein the brain iron deposits are indicative of diseases comprising Alzheimer's disease, Parkinson's disease, Huntington's disease, Hallervorden Spatz disease, other neurodegenerative diseases and atherosclerotic diseases.

7. The method of claim 1 wherein the substantially high magnetic field strength is about 1.5 Tesla (1.5 T) or greater.

8. The method of claim 1 further comprising repeating the acquiring and characterizing steps in at least one successive examination of a given subject for at least one of measuring progression of the disease and measuring response to therapy.

9. A system for detecting iron using magnetic resonance imaging (MRI) comprising:
a magnetic resonance imaging device having a substantially high magnetic field strength and the device being adapted for acquiring magnetic resonance (MR) images by a pulse sequence adapted to create a magnetic field map of the brain for use in enhancing brain iron deposits; and,
an image processor coupled to the imaging device and adapted for characterizing regions of interest using the magnetic field map to detect iron deposits for use in at least one of diagnosis, prognosis, and prediction of progression of iron-dependent diseases.

10. The system of claim 9 wherein the substantially high magnetic field strength is about 1.5 Tesla (1.5 T) and greater.

11. The system of claim 9 wherein the iron-dependent diseases comprise Alzheimer's Disease, Parkinson's Disease, Huntington's Disease, Hallervorden Spatz disease, other neurodegenerative diseases, liver diseases and atherosclerotic diseases.

12. The system of claim 9 wherein the selected pulse sequence is a dual gradient echo pulse sequence and the acquiring step is performed for two different echo times.

13. The system of claim 9 wherein the MR images create a three dimensional phase image of the brain.

14. The system of claim 13 wherein the magnetic field map of the brain is created by fitting a spherical harmonic series to the three dimensional phase image of the brain.

15. The system of claim 14 further comprises subtracting the spherical harmonic series from the magnetic field map to provide a measure of local variation in the magnetic field in the brain.
